(19) United States
(12) Patent Application Publication
(10) Pub. No.: US 2005/0000516 A1
(43) Pub. Date: Jan. 6, 2005

(54) Transportable diving system

(75) Inventors: Benoit Saillet, Albens (FR); Manuel Roure, Gresy sur Aix (FR)

Correspondence Address: GREENBLUM & BERNSTEIN, P.L.C., 1950 ROLAND CLARKE PLACE, RESTON, VA, 20191, US

(73) Assignee: SALOMON S.A., Metz-Tessy (FR)

(21) Appl. No.: 10/896,280

(22) Filed: Jul. 22, 2004

Related U.S. Application Data
(63) Continuation of application No. PCT/FR03/00110, filed on Jan. 14, 2003.

(30) Foreign Application Priority Data
Feb. 1, 2002 (FR) 02.01527

(51) Int. Cl. A61M 16/00

(52) U.S. Cl. 128/201.27

(57) ABSTRACT

An underwater diving system including at least one breathable air tank that remains at the water surface, the air tank being flexible and integrated into a bag, such as a backpack. The flexible air tank is entirely contained within the bag when the air tank is empty, and is partially deployed outside the bag when the air tank is inflated with breathable air.

### CROSS-REFERENCE TO RELATED APPLICATION

This application is a continuation of International Patent Application No. PCT/FR03/00110, having an international filing date of Jan. 14, 2003, the disclosure of which is hereby incorporated by reference thereto in its entirety, and the priority of which is hereby claimed under 35 U.S.C. §120.

This application is based upon French Patent Application No. 02.01527, filed Feb. 1, 2002, the disclosure of which is hereby incorporated by reference thereto in its entirety and the priority of which is hereby claimed under 35 U.S.C. §119.

### BACKGROUND OF THE INVENTION

1. Field of the Invention

The invention relates to underwater diving systems that supply breathable air to a submerged diver from an assembly, including an air tank, that remains at the water surface.

2. Description of Background and Relevant Information

In systems of the aforementioned type, commonly referred to as “hookah diving systems,” the diver is connected to the tank of the air supply assembly at the water surface by a supply circuit having a hose that is generally relatively long to enable the diver to move in a certain zone around and beneath the tank. Unlike the diving systems in which the diver takes the air tank with him under water, the diver using a hookah system only takes under water the hose whose free end is provided with a mouthpiece or a facemask.

There are various types of air tanks for the hookah systems. Some of them are rigid, others are flexible, Among the advantages of the flexible tanks is the fact that the tank is lightweight, and that it can be made very compact when the system is not in use. This makes it easier in particular to transport the system to the diving site.

The principle of a hookah system having a flexible tank is described, for example, in the document GB-2.164.259. A similar system is described in the document EP-A-437.948.

However, the systems described in these documents do not fully take advantage of the flexibility of the tank, and nothing is provided to facilitate the transportation of the system.

### SUMMARY OF THE INVENTION

Therefore, an object of the present invention is to propose a diving system that is simple, practical, and compact, and which is easy to carry. Moreover, it is desired that the tank keep afloat at the surface, even at the end of the dive, not only to prevent the diving system from sinking, but particularly to constitute a buoy on which the diver can be supported at the end of the dive.

The invention therefore proposes an underwater diving system comprising a surface tank for supplying breathable air to the diver, the air tank being flexible and integrated into a bag, the air tank being entirely contained in the bag when it is empty, and the tank being partially deployed outside the bag when it is filled with breathable air.

### BRIEF DESCRIPTION OF THE DRAWINGS

Other characteristics of the invention will become apparent upon reading the detailed description that follows, with reference to the annexed drawings, in which:

FIGS. 1 and 2 are schematic perspective views showing the diving system in use, when it is in its deployed state, FIG. 1 showing the diver being supported by the diving system, and FIG. 2 showing the diver swimming beneath the diving system;

FIG. 3 is a schematic perspective view showing the diving system when the tank is completely folded within a backpack of the system, the system being accompanied by accessories that can be stored inside the bag;

FIGS. 4 and 5 are schematic cross-sectional views showing the deployed and folded positions, respectively; and

FIG. 6 is a schematic view showing a spring-loaded check valve preventing the diver front completely deflating the tank.

### DETAILED DESCRIPTION OF THE INVENTION

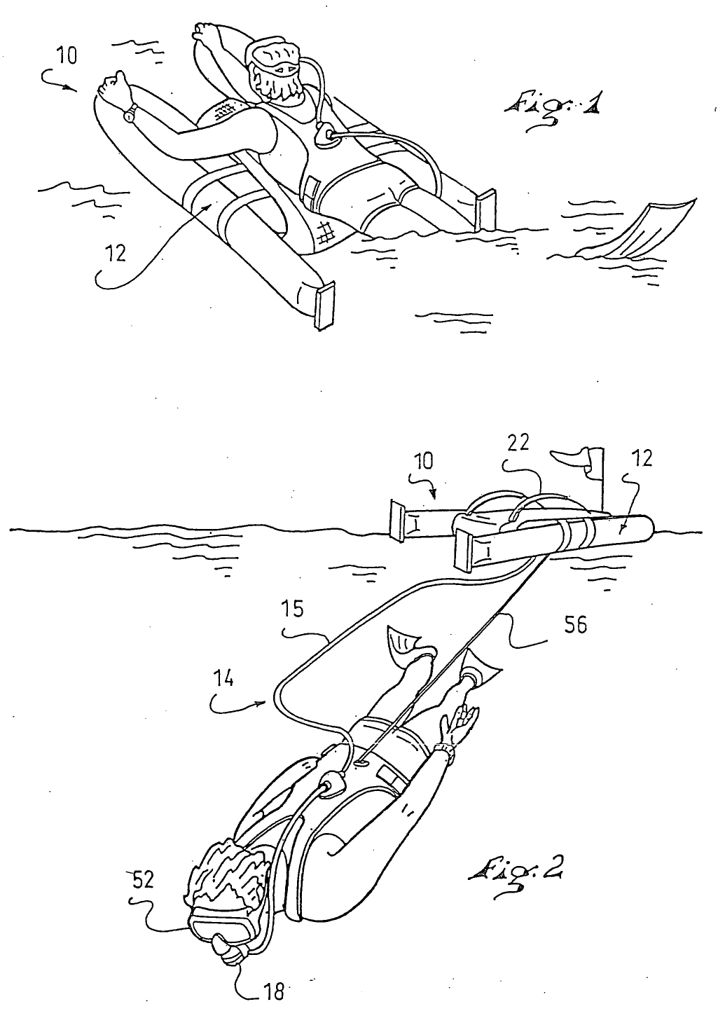

The drawing figures show a diving system 10 comprising a tank 12 for containing pressurized air and comprising an air supply circuit 14 which enables a submerged diver to breathe the air contained in the tank 12. According to the invention, the tank is a flexible tank that comprises, in this case, a flexible tube closed at both of its ends, and it is adapted to remain at the water surface. Due to the ability of the tank to float when it is filled, the diving system forms a float that is capable of bearing at least a portion of the diver's weight (see FIG. 1), which can considerably aid the diver in swimming on the water surface to the exact location of his dive.

The supply circuit 14 is schematically represented by a supply hose 15 that is connected to the tank; the free end of the hose being provided with a mouthpiece 18 preferably incorporating a reducing valve through which the diver can draw the breathable air contained in the tank 12. When the diver goes underwater (see FIG. 2), the tank remains above him at the surface.

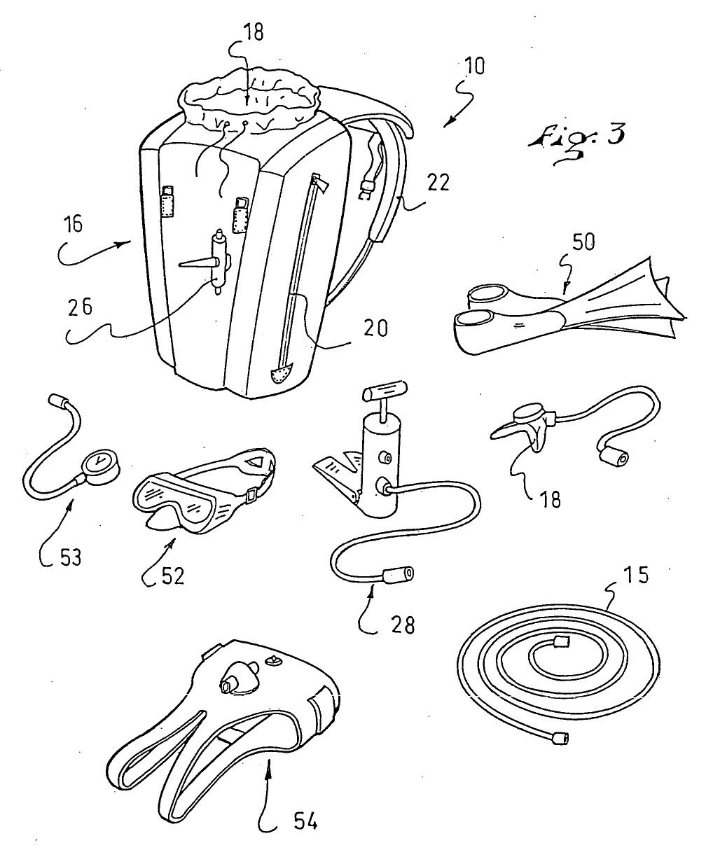

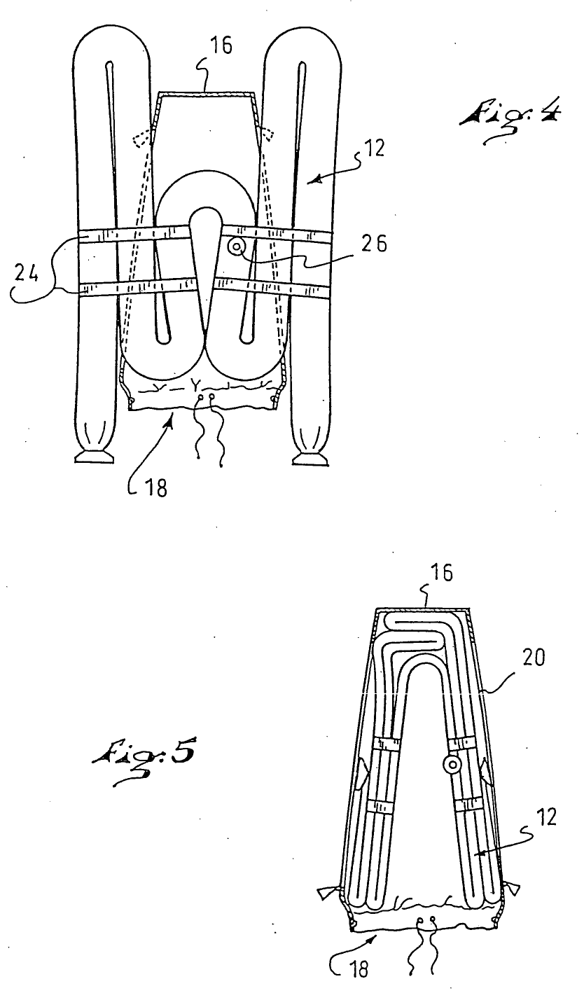

According to the invention, when the tank is filled with pressurized air, it is in a deployed state as shown in FIGS. 1, 2, and 4. When it is empty, the flexible tank can be folded so as to be completely housed in a bag 16 that is an integral part of the diving system. The bag 16 is shown more particularly in FIG. 3, where one can see that takes the form of a flexible backpack of conventional construction demarcating an inner compartment which can be accessed through an upper opening 18, and two lateral openings 20 that are closed, for example, by a zipper or other slide fastener. The bag 16 is further provided with carrying means comprising shoulder straps 22, for example.

As can be seen in FIG. 5, the tube forming the tank 12 is entirely contained in the bag when it is folded. When the tank is supplied with pressurized air, it has a main portion that remains inside the main compartment demarcated by the bag, and end portions corresponding to the two ends of the tube, which extend outside the bag through lateral openings 20 which are opened before the tank is inflated.

In the example shown, the tank is shaped by tightening straps 24 which impart a particular shape to the tube once it is under pressure, so that it is not deployed along a straight line. Thus, in the drawing figures, the central portion of the tank has the shape of an inverted U having two arms, and the two end portions are circled by straps, for example, so as to form a hair-pin that is juxtaposed to one of the arms of the main portion. Thus circled, the tank 12 is deployed substantially in a plane such that it has great stability on the water. Advantageously, the end portions are deployed on each side of the bag in the manner of two parallel longitudinal floats, substantially in the plane of the bag. Conversely, the central portion of the tube forming the tank is preferably permanently attached inside the bag.

As can be seen in the drawing figures, the tank comprises an inlet-outlet valve 26 which, in the example shown, is connected to the central portion of the tube forming the tank, and which extends outside the bag through an opening provided for this purpose, on a front surface opposite a rear surface on which the carrying means 22 are arranged. In this way, as can be seen in FIG. 1, the diving system is provided so that, when the tank 12 and its bag 16 float at the water surface, the carrying means are arranged on the emerged surface of the system, whereas the inlet-outlet valve 26 is arranged on the submerged surface of the system. In this way, the connection of the hose 15 of the supply circuit 14 to the tank 12 is on the lower side of the floating assembly formed by the tank and the bag. Thus, a complete stability of the assembly is guaranteed since the floating assembly cannot be caused to turn over if traction is exerted on the supply hose 15.

The diving system 10 also can include means (not shown) which allow the tank to be filled with a gaseous breathing mixture, such as air. These means can be a mere device for coupling to an air compressor. It can also be means adapted to be carried with the tank. For example, air is stored in the tank 12 under a maximum pressure between 4 and 20 bars.

Preferably, the tank nonetheless has a maximum inextensible volume greater than 20 liters, which volume is reached, for example, at an absolute pressure on the order of 1.2-1.5 bars. Beginning at this pressure, the tank has reached its “inflated” state and, beyond, its volume can no longer increase significantly. Conversely, the tank preferably has a very small external volume once it is “deflated.” Advantageously, the tank is sized so that its maximum volume is on the order of 40-50 liters, and so that it resists pressures on the order of 4 or 5 bars. In the example shown, the tube forming the tank is made of a woven reinforcement that is coated or embedded in a flexible, impervious matrix, for example, a polyurethane matrix. The constituent material of the tank can also be a flexible and impervious plastic material, or the tank can be constructed with a multilayered material having a first layer of material that ensures the mechanical resistance to pressure, and at least one impervious inner layer.

In the example shown in FIG. 3, it is provided that the diving system comprise a device for filing up the tank, for example, a motorized pump, or a human-actuated pump, such as a manual pump 28. This pump 28 can thus be stored in the backpack, at least when the tank in the folded configuration.

The bag also makes it possible to carry other accessories, whether necessary or only useful for using the diving system, such as flippers 50, a mask 52, a pressure gauge 53, or yet a harness 54 provided with a rope 56 by means of which the diver can tow the tank remaining at the surface without exerting any traction force on the air supply hose (see FIG. 2).

In addition to the main compartment in which the tank is housed, the backpack 16 can comprise auxiliary compartments, for example, impervious/waterproof compartments. It can also have a rigid or semi-rigid structure that can contribute to the comfort of the bag.

In any case, the advantage is to be understood in the float conserving its full floating capability, or a least a substantial portion thereof.

Thus, according to the invention, the diving system 10 comprises a member 30 for shutting off the supply circuit 14 which, under a predetermined pressure threshold in the tank 12, prevents the diver from retrieving air from the tank. The predetermined pressure threshold is determined as a function of the pressure that is necessary for maintaining the tank 12 in an “inflated” state in which it conserves all, or at least a substantial portion of its floating capability.

This predetermined shut-off threshold can be, for example, an absolute pressure threshold in the tank. It can thus be set at an absolute pressure value comprised between 1.2 and 1.5 bars. It can also be a relative pressure threshold in the tank with respect to the atmospheric pressure, the shut-off member imparting a pressure in the tank that is greater than the atmospheric pressure, for example, 50-500 millibars.

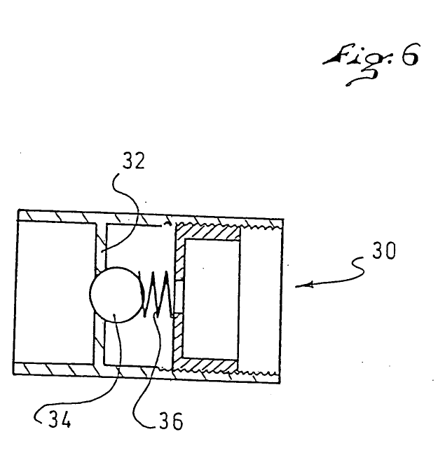

FIG. 6 shows an example of a shut-off member that is capable of being used to implement the invention. The cutoff member 30 here is a spring-loaded check valve made in the form of a ball spring-loaded check valve, a member that is known in itself, and it is shown in the position for shutting off the supply.

The valve 30 is, for example, integrated into the inlet-outlet valve 26. It comprises a seat 32 on which a ball 34, arranged on the downstream side of the threshold 32, is pressed upstream by a compression spring 36.

When, on both sides of the ball 34, the force of air pressure in the tank 12 is greater than the sum of the pressure in the supply hose (i.e., the pressure in the diver's lungs when he seeks to breathe in) and of the prestress of the spring 34, the ball 34 is separated from the seat 32 and air can circulate from the tank 12 toward the supply hose 16, therefore toward the diver. Conversely, as soon as the air pressure in the tank 12 drops below said sum, the ball 34 is pressed by the spring 36 against the seat 32, which interrupts the circulation of air, as shown in FIG. 2. The diver can no longer continue to empty the tank; the air pressure within the tank therefore cannot drop below a threshold value, so that the tank cannot be deflated and maintains its floating capability. In this embodiment, the threshold pressure is an absolute pressure that varies as a function of the depth at which the diver is located at the given moment. Indeed, the pressure in the supply hose is geater than the atmospheric pressure, and the value of the over-pressure is proportional to the depth of the diver. Advantageously, the value of this absolute pressure decreases progressively as the diver swims back up to the surface, the latter being able again to breathe a little bit of air after swimming back up over a certain distance.

The embodiment of the shut-off member which is proposed here is only a non-limiting example. Other known shut-off members fulfilling the same function could be used for implementing the invention.

1. An underwater diving system comprising:
a bag having at least one interior compartment;
at least one air tank adapted to be filled with breathable air for a diver;
the air tank being flexible and is integrated into the at least one interior compartment of the bag, the bag and air tank being adapted to remain at a water surface above a submerged diver;
the air tank being configurable to be entirely contained within the bag when the air tank is empty;
the air tank being configurable to be partially deployed outside the bag when the air tank is filled with breathable air.

2. An underwater diving system according to claim 1, wherein:
the air tank comprises a flexible tube having at least one shaped portion.

3. An underwater diving system according to claim 2, wherein:
the shaped portions of the air tank are maintained in a predeterminate folded shape to prevent the flexible tube from completely unfolding when air fills the flexible tube.

4. An underwater diving system according to claim 1, wherein:
the air tank comprises a main portion remaining within the bag when the air tank is filled.

5. An underwater diving system according to claim 1, wherein:
the air tank, when filled, has a volume greater than 20 liters.

6. An underwater diving system according to claim 1, wherein:
the bag comprises a backpack having adequate capacity to contain, in addition to the air tank, accessories of the underwater diving system.

7. An underwater diving system according to claim 6, wherein:
the accessories comprise an air supply circuit and/or a mouthpiece and/or a pump for filling the air tank.

8. An underwater diving system according to claim 6, wherein:
the accessories comprise a manually actuated pump for filling the air tank.

9. An underwater diving system according to claim 1, further comprising:
an air supply circuit for supplying a diver with air from the flexible air tank;
a member for shutting off the supply of air to the diver from the flexible air tank in response to air pressure within the air tank dropping below a predetermined shut-off threshold.

10. An underwater diving system according to claim 9, wherein:
the shut-off threshold is a predetermined magnitude for maintaining the flexible air tank in an inflated state.

11. An underwater diving system according to claim 9, wherein:
the shut-off member is positioned in the air supply circuit.

12. An underwater diving system according to claim 11, wherein:
the shut-off member is a valve.

13. An underwater diving system according to claim 12, wherein:
the valve is a check valve comprising a shutter pressed, upstream, against a shutting seat by an elastic return means that prevent opening of the check valve in response to air pressure in the air tank being below the predetermined threshold.

14. An underwater diving system according to claim 1, wherein:
the bag is a backpack having at least one shoulder strap.
